(19) United States
(12) Patent Application Publication
(10) Pub. No.: US 2015/0367015 A1
(43) Pub. Date: Dec. 24, 2015

(54) REMOVABLE SPRAY SORBENT ARTICLE

(71) Applicant: Mark Backus, Owego, NY (US)

(72) Inventor: Mark Backus, Owego, NY (US)

(21) Appl. No.: 14/309,536

(22) Filed: Jun. 19, 2014

(51) Int. Cl. A61L 9/12

(52) U.S. Cl. CPC A61L 9/12

(57) ABSTRACT

An adjustable spray sorbent article for receiving aerosol spray and collecting the liquids and solids suspended therein. The spray sorbent article comprises a base article defined as a semi-permeable towel configured to enable solid particles or liquid droplets in an aerosol mixture to be caught while the air carrying such particles passes through and at least one of various attachment components, typically selected based on the target location for the base article. The base article is adapted so that when an aerosol spray is directed towards the base article, substantially all of the suspended particles, including all active ingredients therein as well as the propellants are captured while the suspending gas passes through. Through this action, the base article prevents such particles from accumulating on surfaces on being inhaled in mass by users, while still allowing fragrant particles to be emitted into the surrounding air more slowly.

### BACKGROUND OF THE INVENTION

1. Field of the Invention

This invention relates generally to fragrance accessories and, more particularly, to a washable towel configured to attach fragrant aerosol emissions and emit fragrance therefrom.

2. Description of the Prior Art

The use of aerosol sprays for the distribution of fragrance in a room is well known. A problem which still exists, however, is that simply spraying a fragrant aerosol around in the air in order to disperse the fragrance is typically accompanied by the dispersal of harmful hydrocarbon propellants and/or or the accumulation of reside on surfaces in the area. Thus, there remains a need for a spray sorbent article onto which such fragrant sprays can be directed in order to avail their aroma to the immediate vicinity. It would be helpful if such a spray sorbent article was adapted retain and emit fragrances in the sprays directed thereon. It would be additionally desirable for such a spray sorbent article was adapted to be attached, hung or otherwise placed in a desired area where it could receive fragrant spray.

The Applicant's invention described herein provides for a spray sorbent article which receives fragrance from an aerosol spray and emits it in the vicinity thereof. The primary components in Applicant's spray sorbent article are a base article and an attachment component. When in operation, the spray sorbent article enables fragrances typically distributed from aerosol cans to be received and continually emitted from a desired location in the area desired to freshened. As a result, many of the limitations imposed by prior art structures are removed.

### SUMMARY OF THE INVENTION

A spray sorbent article for receiving aerosol spray and collecting the liquids and solids suspended therein. The spray sorbent article comprises a base article defined as a semi-permeable towel configured to enable solid particles or liquid droplets in an aerosol mixture to be caught while the air carrying such particles passes through and at least one of various attachment components, typically selected based on the target location for the base article. The base article is adapted so that when an aerosol spray is directed towards the base article, substantially all of the suspended particles, including all active ingredients therein as well as the propellants are captured while the suspending gas passes through. Through this action, the base article prevents such particles from accumulating on surfaces on being inhaled in mass by users, while still allowing fragrant particles to be emitted into the surrounding air more slowly.

It is an object of this invention to provide a spray sorbent article onto which such fragrant sprays can be directed in order to avail their aroma to the immediate vicinity.

It is another object of this invention to provide a spray sorbent article adapted retain and emit fragrances in the sprays directed thereon.

It is yet another object of this invention to provide a spray sorbent article adapted to be attached, hung or otherwise placed in a desired area where it could receive fragrant spray.

These and other objects will be apparent to one of skill in the art.

### BRIEF DESCRIPTION OF THE DRAWINGS

FIG. 1a is a front elevational view of a spray sorbent article in accordance with the present invention having a hanging attachment component.

FIG. 1b is a front elevational view of a spray sorbent article in accordance with the present invention having an adhesive attachment component.

FIG. 1c is a front elevational view of a spray sorbent article in accordance with the present invention having a fabric hook and loop attachment component.

FIG. 1d is a front elevational view of a spray sorbent article in accordance with the present invention having a pole attachment component.

### DETAILED DESCRIPTION OF THE INVENTION

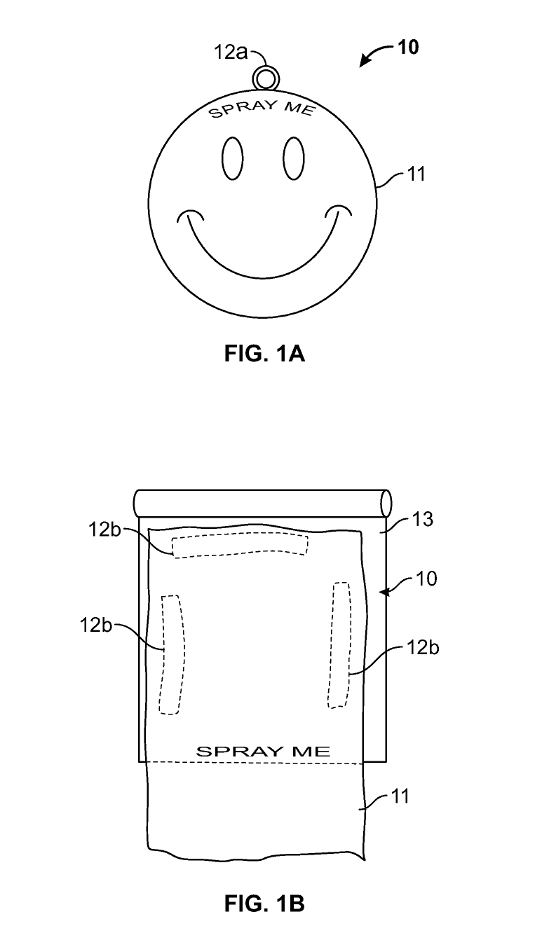

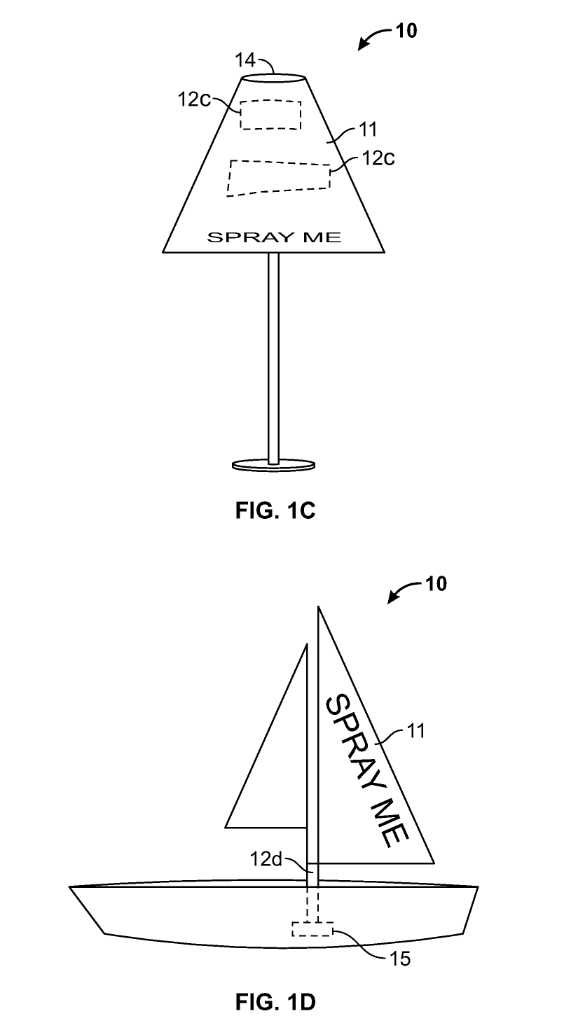

Referring now to the drawings and in particular FIG. 1A, FIG. 1B, FIG. 1C, and FIG. 1D, a spray sorbent article 10 is shown having a base article 11 and an attachment component 12a, 12b, 12c, 12d (collectively, 12). The base article 11 is defined as a semi-permeable towel configured to enable solid particles or liquid droplets in an aerosol mixture to be caught while the air carrying such particles passes through. In this regard, when an aerosol spray is directed towards the base article 11, substantially all of the active ingredients therein, as well as the propellants are captures while the suspending gas passes through. In the preferred embodiment, the towel's semi-permeable nature is due to microscopic perforations in the body thereof, whereby minimal restriction is placed on suspending gas while increased the surface area is available for the solids and liquids in the aerosol to attach to. The material of the towel in the preferred embodiment is defined by an absorbent property, which enables the solids and liquids to enter into the material of the towel. It is contemplated that a fabric such as terrycloth is may be employed. In such an embodiment, it is contemplated that loops may be included on the surface of the terrycloth in order to increase the surface area, and thus, absorption. The use of the absorbed terrycloth as a base article 11 additionally enables the spray sorbent article 10 to be washable.

In an alternate embodiment, the material of the towel is defined by an adsorbent property, which enables the solids and liquids to adhere to the surfaces of the towel.

One of various attachment components 12 may be employed with a base article 11, typically selected based on the target location for the base article 11. In an embodiment intended to be suspended in an area, a hanging attachment component 12a is employed.

In an embodiment intended to be attached to a impermeable material, such as a plastic window shade 13, an adhesive strip 12b is disposed on one side of the base article 11. The adhesive strip 12b enables the base article 11 to be removably fastened to the impermeable material.

In an embodiment intended to be attached to a permeable or semi-permeable material, such as a fabric light shade 14, a fabric hook and loop fastener strip 12c is disposed on one side of the base article 11. In such an embodiment, it is contemplated that the hook portion of the fabric hook and loop fastener would be facing away from the base article 11 so as to attach to the permeable or semi-permeable fabric.

In an embodiment intended to be attached to a structure having a receiving port, such as a flag stand 15, a pole attachment structure 12d is attached to the base article 11.

It is contemplated that the semi-permeable construction of the base article 11 enables ambient air to contact it and pass through. Through this action, fragrance which has been sprayed onto the base article 11 is emitted into the area surrounding the towel.

The instant invention has been shown and described herein in what is considered to be the most practical and preferred embodiment. It is recognized, however, that departures may be made therefrom within the scope of the invention and that obvious modifications will occur to a person skilled in the art.

What is claimed is:

1. A spray sorbent article, comprising:
a semi-permeable base article configured to absorb suspended particles in suspending gas on contact therewith;
an attachment structure connected to said base article, wherein said attachment structure is adapted to removably attach said spray sorbent article to a fixed structure; and
wherein said base article is adapted to emit fragrant suspended particles absorbed from a suspending gas.

2. The spray sorbent article of claim 1, wherein said attachment structure is defined by a hanging attachment component.

3. The spray sorbent article of claim 1, wherein said attachment structure is defined by at least one adhesive body attached to one side of said base article.

4. The spray sorbent article of claim 1, wherein said attachment structure is defined by the hook portion of a fabric hook and loop fastener attached to one side of said base article.

5. The spray sorbent article of claim 1, wherein said attachment structure is defined by an attachment pole attached to said base article.

6. A spray sorbent article, comprising:
a semi-permeable base article configured to adsorb suspended particles in suspending gas on contact therewith;
an attachment structure connected to said base article, wherein said attachment structure is adapted to removably attach said spray sorbent article to a fixed structure; and
wherein said base article is adapted to emit fragrant suspended particles adsorbed from a suspending gas.

7. The spray sorbent article of claim 6, wherein said attachment structure is defined by a hanging attachment component.

8. The spray sorbent article of claim 6, wherein said attachment structure is defined by at least one adhesive body attached to one side of said base article.

9. The spray sorbent article of claim 6, wherein said attachment structure is defined by the hook portion of a fabric hook and loop fastener attached to one side of said base article.

10. The spray sorbent article of claim 6, wherein said attachment structure is defined by an attachment pole attached to said base article.
